SEQUENCE LISTING (4 sequences)

SEQ ID NO: 1 - DNA, 932 bp, Synechocystis PCC 6803
  CDS (4)..(930)
gccatggcaactatccaagctttttggcgcttctcccgcccccataccatcattggtacaactctgagcgtctgggctgtgtatctgttaactattctcggggatggaaactcagttaactcccctgcttccctggatttagtgttcggcgcttggctggcctgcctgttgggtaatgtgtacattgtcggcctcaaccaattgtgggatgtggacattgaccgcatcaataagccgaatttgcccctagctaacggagatttttctatcgcccagggccgttggattgtgggactttgtggcgttgcttccttggcgatcgcctggggattagggctatggctggggctaacggtgggcattagtttgattattggcacggcctattcggtgccgccagtgaggttaaagcgcttttccctgctggcggccctgtgtattctgacggtgcggggaattgtggttaacttgggcttatttttattttttagaattggtttaggttatccccccactttaataacccccatctgggttttgactttatttatcttagttttcaccgtggcgatcgccatttttaaagatgtgccagatatggaaggcgatcggcaatttaagattcaaactttaactttgcaaatcggcaaacaaaacgtttttcggggaaccttaattttactcactggttgttatttagccatggcaatctggggcttatgggcggctatgcctttaaatactgctttcttgattgtttcccatttgtgcttattagccttactctggtggcggagtcgagatgtacacttagaaagcaaaaccgaaattgctagtttttatcagtttatttggaagctatttttcttagagtacttgctgtatcccttggctctgtggttacctaatttttctaatactattttttaggg

SEQ ID NO: 2 - protein, 308 aa, Synechocystis PCC 6803
MATIQAFWRFSRPHTIIGTTLSVWAVYLLTILGDGNSVNSPASLDLVFGAWLACLLGNVYIVGLNQLWDVDIDRINKPNLPLANGDFSIAQGRWIVGLCGVASLAIAWGLGLWLGLTVGISLIIGTAYSVPPVRLKRFSLLAALCILTVRGIVVNLGLFLFFRIGLGYPPTLITPIWVLTLFILVFTVAIAIFKDVPDMEGDRQFKIQTLTLQIGKQNVFRGTLILLTGCYLAMAIWGLWAAMPLNTAFLIVSHLCLLALLWWRSRDVHLESKTEIASFYQFIWKLFFLEYLLYPLALWLPNFSNTIF

SEQ ID NO: 3 - DNA, 26 bp, Artificial Sequence
  primer_bind (1)..(26)
  Description of artificial sequence primer
ggatccgccatggcaactatccaagc

SEQ ID NO: 4 - DNA, 25 bp, Artificial Sequence
  primer_bind (1)..(25)
  Description of artificial sequence primer
ggatccccctaaaaaatagtattag

(19) United States
(12) Patent Application Publication
(10) Pub. No.: US 2003/0084479 A1
(43) Pub. Date: May 1, 2003

(54) Homogentisate phytyl transferase

(76) Inventors: Karin Herbers, Quedlinburg (DE); Ralf Badur, Goslar (DE); Irene Kunze, Gatersleben (DE); Susanne Sommer, Quedlinburg (DE); Rainer Lemke, Quedlingburg (DE); Michael Geiger, Quedlinburg (DE)

Correspondence Address: Nixon & Vanderhye, 8th Floor, 1100 North Glebe Road, Arlington, VA, 22201-4714, US

(21) Appl. No.: 10/204,700

(22) PCT Filed: Feb. 16, 2001
(86) PCT No.: PCT/EP01/01720

(30) Foreign Application Priority Data
Feb. 25, 2000 (DE) 10009002.8

(51) Int. Cl. A01H 5/00; C12N 9/10; C12N 15/82; C12N 5/04

(52) U.S. Cl. 800/281; 435/419; 435/193; 435/320.1

(57) ABSTRACT

The invention relates to nucleic acid sequences encoding a protein with homogentisate phytyltransferase activity, to the use of the nucleic acids for generating transgenic organisms such as, for example, transgenic plants with an elevated tocopherol and tocotrienol content, to a method of generating plants with an elevated tocopherol and/or tocotrienol content, and to the transgenic plants themselves.

The invention relates to nucleic acid sequences encoding a protein with homogentisate phytyltransferase activity, to the use of the nucleic acids for generating transgenic organisms, such as, for example, transgenic plants with an elevated tocopherol and/or tocotrienol content, to a method for generating plants with an elevated tocopherol and tocotrienol content, and to the transgenic organisms, such as, for example, transgenic plants, themselves.

The naturally occurring eight compounds with vitamin E activity are derivatives of 6-chromanol (Ullmann's Encyclopedia of Industrial Chemistry, Vol. A 27 (1996), VCH Verlagsgesellschaft, Chapter 4., 478-488, Vitamin E). The tocopherol group (1a-d) has a saturated side chain, while the tocotrienol group (2a-d) has an unsaturated side chain:

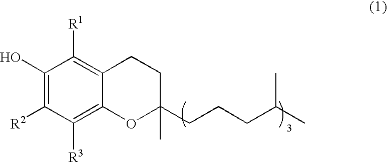

1a, α-tocopherol: R¹=R²=R³=CH₃

1b, β-tocopherol [148−03−8]: R¹=R³=CH₃, R²=H

1c, γ-tocopherol [54−28−4]: R¹=H, R²=R³=CH₃

1d, δ-tocopherol [119−13−1]: R¹=R²=H, R³=CH₃

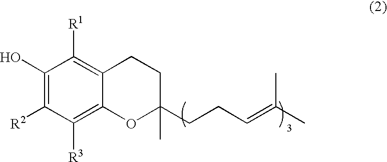

2a, α-tocotrienol [1721−51−3]: R¹=R²=R³=CH₃

2b, β-tocotrienol [490−23−3]: R¹=R³=CH₃, R²=H

2c, γ-tocotrienol [14101−61−2]: R¹=H, R²=R³=CH₃

2d, δ-tocotrienol [25612−59−3]: R¹=R²=H, R³=CH₃

For the purposes of the present invention vitamin E is to be understood as meaning all of the eight abovementioned tocopherols and tocotrienols with vitamin E activity.

These compounds with vitamin E-activity are important natural lipid-soluble antioxidants. Vitamin E deficiency leads to pathophysiological situations in humans and animals. Thus, vitamin E compounds are of great economic value as additives in the food and feed sectors, in pharmaceutical formulations and in cosmetic applications.

An economical method for producing vitamin E compounds and foodstuffs and animal feeds with an increased vitamin E content are therefore very important.

Especially economical methods are biotechnological methods which exploit the proteins and biosynthesis genes of tocopherol or tocotrienol biosynthesis from vitamin E-producing organisms.

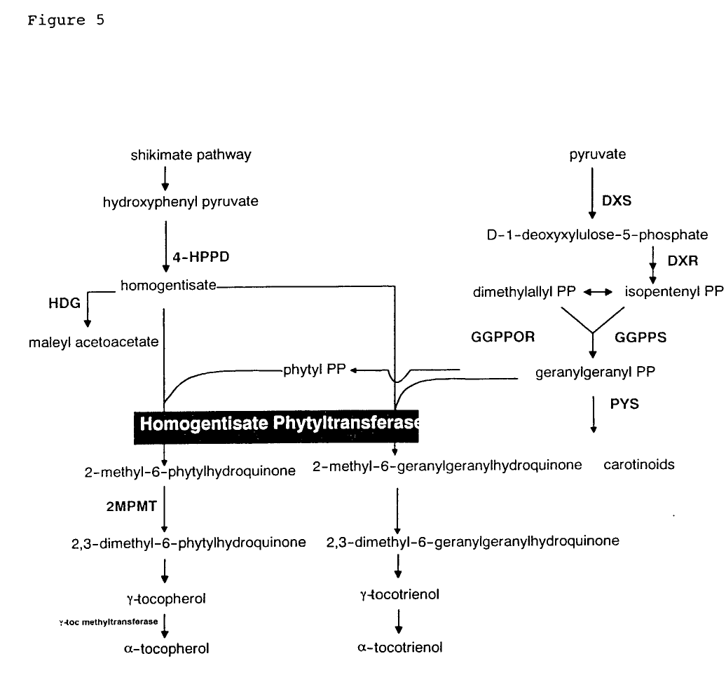

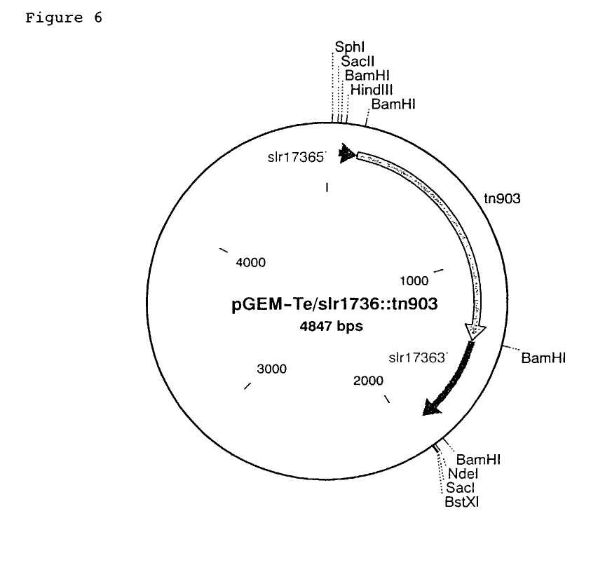

FIG. 5 shows a biosynthesis scheme of tocopherols and tocotrienols.

During biosynthesis, homogentisic acid (homogentisate) is bound to phytyl pyrophosphate (PPP) or geranylgeranyl pyrophosphate in order to form the precursors of α-tocopherol and α-tocotrienol, which are 2-methylphytylhydroquinone and 2-methylgeranyl-geranylhydroquinone, respectively. Methylation steps with S-adenosylmethionine as methyl donor first gives 2,3-dimethyl-6-phytylhydroquinone, cyclization then gives γ-tocopherol, and further methylation gives α-tocopherol.

Katani et al., Annu. Rev. Plant Physiol. Plant Mol. Biol, 1998, 49, 151 to 157 describe the full genomic sequence of the cyanobacterium Synechocystis sp. PCC6803.

Little is known as yet about increasing the metabolite flow to increase the tocopherol or tocotrienol content in transgenic organisms, for example in transgenic plants, by overexpressing individual biosynthesis genes.

WO 97/27285 describes a modification of the tocopherol content by increased expression or by downregulation of the enzyme p-hydroxyphenylpyruvate dioxygenase (HPPD).

WO 99/04622 describes gene sequences encoding a γ-tocopherol methyltransferase from Synechocystis PCC6803 and Arabidopsis thaliana and its incorporation into transgenic plants.

WO 99/23231 shows that the expression of a geranylgeranyl reductase in transgenic plants results in an increased tocopherol biosynthesis.

It is an object of the present invention to provide a further biosynthesis of the vitamin E biosynthetic pathway and thus further advantageous transgenic plants with an elevated tocopherol and tocotrienol content.

We have found that this object is achieved by finding nucleic acid sequences encoding a homogentisate phytyltransferase and by overexpressing the homogentisate phytyltransferase gene in plants.

Accordingly, the present invention relates to proteins which have the activity of a homogentisate phytyltransferase (HGPT)that is to say the ability of binding phytyl pyrophosphate to homogentisate, that is to say which have, for example, an enzymatic activity for converting homogentisate and phytyl pyrophosphate into 2-methylphytylhydroquinone.

Preferred 2-methylphytylhydroquinones are 2-methyl-6-phytyl-hydroquinone or 2-methyl-5-phytylhydroquinone.

Homogentisate phytyltransferases are to be understood as meaning in the following context the proteins according to the invention.

Preferred proteins have the enzymatic activity for converting homogentisate and phytylpyrophosphate into 2-methylphytyl-hydroquinone and comprise the amino acid sequence SEQ ID NO. 2 or a sequence which is derived from this sequence by substitution, insertion or deletion of amino acids which has at least 20%, preferably 40%, by preference at least 60%, more preferably at least 80%, especially preferably at least 90% homology at the amino acid level with the sequence SEQ ID NO. 2.

Further examples of the proteins according to the invention can readily be found, for example, in various organisms whose genomic sequence is known such as, for example, Arabidopsis thaliana, by homology comparison of the amino acid sequences or of the corresponding backtranslated nucleic acid sequences from databases with SEQ ID. NO. 2.

The proteins according to the invention can be used as homogentisate phytyltransferases.

The preferred proteins are preferred for all of the uses according to the invention of the proteins according to the invention.

Substitution is to be understood as meaning the exchange of one or more amino acids by one or more amino acids. Preferably, so-called conservative exchanges are carried out in which the amino acid which is replaced has a similar property as the original amino acid, for example the exchange of Glu by Asp, Gln by Asn, Val by Ile, Leu by Ile, Ser by Thr.

A deletion is the replacement of an amino acid by a direct bond. Preferred positions for deletions are the termini of the polypeptide and the linkages between the individual protein domains.

Insertions are introductions of amino acids into the polypeptide chain, a direct bond formally being replaced by one or more amino acids.

Homology between two proteins is to be understood as meaning the identity of the amino acids over in each case the entire length of the protein which is calculated by comparison with the aid of the program algorithm GAP (UWGCG, University of Wisconsin, Genetic Computer Group) setting the following parameters:

| | |
|---|---|
| Gap Weight: | 12 |
| Length Weight: | 4 |
| Average Match: | 2.912 |
| Average Mismatch: | −2.003 |

Accordingly, a protein which has at least 20% homology at the amino acid level with the sequence SEQ ID NO. 2 is to be understood as meaning a protein which, upon comparison of its sequence with the sequence SEQ ID No. 2 using the above program algorithm with the above parameter set has at least 20% homology.

The homogentisate phytyltransferases according to the invention are capable of converting homogentisate derivatives and phytyl pyrophosphate derivatives into 2-methylphytylhydroquinone derivatives and/or of converting homogentisate derivatives and geranylgeranyl pyrophosphate derivatives into 2-methylgeranyl-geranylhydroquinone derivatives.

Homogentisate derivatives are to be understood as meaning homogentisate and homogentisate compounds derived therefrom which are accepted as substrates by the homogentisate phytyl-transferases according to the invention.

Phytyl pyrophosphate derivatives are to be understood as meaning phytyl pyrophosphate and phytyl pyrophosphate compounds derived therefrom which are accepted as substrates by the homogentisate phytyltransferases according to the invention.

Accordingly, 2-methylphytylhydroquinone derivatives are to be understood as meaning the resulting compounds of the enzymatic conversion such as, for example, 2-methylphytylhydroquinone and the corresponding derived compounds.

Preferred 2-methylphytylhydroquinone derivatives are derivatives of 2-methyl-6-phytylhydroquinone or 2-methyl-5-phytyl-hydroquinone.

Geranylgeranyl pyrophosphate derivatives are to be understood as meaning geranylgeranyl pyrophosphate and geranylgeranyl pyrophosphate compounds derived therefrom which are accepted as substrates by the homogentisate phytyltransferases according to the invention.

Accordingly, 2-methylgeranylgeranylhydroquinone derivatives are to be understood as meaning the resulting compounds of the enzymatic conversion such as, for example, 2-methylgeranyl-geranylhydroquinone and the corresponding derived compounds.

Preferred 2-methylgeranylgeranylhydroquinones are 2-methyl-6-geranylgeranylhydroquinone or 2-methyl-5-geranyl-geranylhydroquinone.

Preferred 2-methylgeranylgeranylhydroquinone derivatives are derivatives of 2-methyl-6-geranylgeranylhydroquinone or 2-methyl-5-geranylgeranylhydroquinone.

Accordingly, the invention relates to a biotransformation method, which comprises converting homogentisate derivatives and phytyl pyrophosphate derivatives into 2-methylphytylhydroquinone derivatives or homogentisate derivatives and geranylgeranyl pyrophosphate derivatives into 2-methylgeranylgeranylhydroquinone derivatives in the presence of a homogentisage phytyltransferase according to the invention.

In principle, the biotransformation can be carried out with intact cells which express the enzyme HGPT or cell extracts from these cells or else with purified or ultrapure HGPT. The homogentisate phytyltransferase may also exist in free or in immobilized form in this context.

Furthermore, the homogentisate phytyltransferases according to the invention can be used for the production of vitamin E. The enzymatic biosynthesis step of the homogentisate phytyl-transferases can be carried out in vitro or as described hereinbelow in vivo, for example in transgenic organisms, such as, for example, in transgenic plants.

Accordingly, the invention relates to a process for the production of vitamin E, wherein homogentisate derivatives and phytyl pyrophosphate derivatives are converted into 2-methylphytylhydroquinone derivatives or homogentisate derivatives and geranylgeranyl pyrophosphate derivatives are converted into 2-methylgeranylgeranylhydroquinone derivatives in the presence of the homogentisate phytyltransferase according to the invention.

Furthermore, the biosynthetic pathway of vitamin E offers target enzymes for the development of inhibitors. Since, according to the present-day state of the art, no enzyme exists in human and animal organisms which is identical with, or similar to, the Synechocystis HGPT, it can be assumed that inhibitors act highly specifically on plants.

The invention therefore also relates to the use of the homogentisate phytyltransferase according to the invention as herbicide target for finding homogentisate phytyltransferase inhibitors.

HGPT is a target for herbicides. To be able to find effective HGPT inhibitors, it is necessary to provide suitable assay systems with which inhibitor-enzyme binding studies can be carried out. To this end, the complete Synechocystis HGPT cDNA sequence, for example, is cloned into an expression vector (pQE, Qiagen) and overexpressed in E. coli.

The HGPT protein expressed with the aid of the expression cassette according to the invention is particularly suitable for finding HGPT-specific inhibitors.

Accordingly, the invention relates to a method for finding homogentisate phytyltransferase inhibitors, wherein the enzymatic activity of the homogentisate phytyltransferase is measured in the presence of a chemical compound and, when the enzymatic activity is reduced in comparison with the uninhibited activity, the chemical compound constitutes an inhibitor.

To this end, the HGPT can be employed for example in an enzyme assay in which the HGPT activity is determined in the presence and absence of the active ingredient to be tested. A qualitative and quantitative statement on the inhibitory behavior of the active ingedient to be tested can be made by comparing the two activity determinations.

The assay system according to the invention allows a multiplicity of chemical compounds to be tested rapidly and simply for herbicidal properties. The method allows the reproducible selection, from a large number of substances, specifically of those which are highly effective in order subsequently to carry out, with these substances, other in-depth tests with which the skilled worker is familiar.

The invention therefore furthermore relates to herbicidal active ingredients which can be identified using the above-described assay system.

The homogentisate phytyltransferases according to the invention can be prepared as described hereinbelow by gene expression of the corresponding nucleic acids encoding these proteins from natural or genetically modified organisms.

The invention furthermore relates to nucleic acids, termed homogentisate phytyltransferase genes (HPGT genes) hereinbelow which encode the proteins according to the invention described hereinabove.

The nucleic acid sequence can be, for example, an RNA, DNA or cDNA sequence. Coding sequences which are suitable for insertion into a nucleic acid construct such as, for example, an expression cassette, are, for example, those which encode an HGPT and which impart to the host the ability of overexpressing tocopherols and/or tocotrienols.

Suitable nucleic acid sequences can be obtained by backtranslating the polypeptide sequence in accordance with the genetic code.

Codons which are preferably used for this purpose are those which are used frequently in accordance with the codon usage which is specific to the organism. The codon usage can be determined readily with the aid of computer evaluations of other common known genes of the organism in question.

If, for example, the protein is intended to be expressed in a plant, it is frequently advantageous to use the codon usage of the plant for backtranslation.

Preferred nucleic acids encode a plant homogentisate phytyltransferase or a homogentisate phytyltransferase from cyanobacteria.

An especially preferred nucleic acid has the sequence SEQ ID NO. 1. This nucleic acid constitutes a prokaryotic genomic DNA from the cyanobacterium Synechocystis sp. PCC6803 which encodes the homogentisate phytyltransferase of the sequence SEQ ID NO. 2.

All the abovementioned homogentisate phytyltransferase genes can be prepared in a manner known per se by chemical synthesis from the nucleotide units, such as, for example, by fragment condensation of individual overlapping complementary nucleic acid units of the double helix. The chemical synthesis of the oligonucleotides can be carried out, for example, in the known manner using the phosphoamidite method (Voet, Voet, 2^{nd} Edition, Wiley Press New York, pages 896-897). The addition of synthetic oligonucleotides and the filling-in of gaps with the aid of the Klenow fragment of the DNA polymerase and ligation reactions and general cloning methods are described in Sambrook et al. (1989), Molecular cloning: A laboratory manual, Cold Spring Harbor Laboratory Press.

The invention furthermore relates to the use of the HGPT according to the invention or of the HGPT genes according to the invention for the production of antibodies.

The invention furthermore relates to nucleic acid constructs comprising one of the above-described homogentisate phytyltransferase genes according to the invention which are linked functionally to one or more regulatory signals which ensure transcription and translation in prokaryotic or eukaryotic organisms.

These regulatory sequences are, for example, sequences to which inductors or repressors bind, thus regulating expression of the nucleic acid. In addition to these novel regulatory sequences, or instead of these sequences, the natural regulation of these sequences before the actual structural genes may still be present and, if appropriate, may have been genetically modified so that the natural regulation has been switched off and gene expression increased. However, the nucleic acid construct can also have a simpler structure, that is to say no additional regulatory signals are inserted before the abovementioned homogentisate phytyltransferase genes, and the natural promoter with its regulation is not removed. Instead, the natural regulatory sequence is mutated in such a way that regulation no longer takes place and gene expression is increased. These modified promoters can also be placed before the natural genes by themselves in order to increase activity.

In addition, the nucleic acid construct may advantageously also comprise one or more so-called enhancer sequences functionally linked to the promoter, which makes possible the increased expression of the nucleic acid sequence. Additional advantageous sequences may also be inserted at the 3′ end of the DNA sequences, such as further regulatory elements or terminators. The abovementioned homogentisate phytyltransferase genes may be present in the gene construct in the form of one or more copies.

Nucleic acid constructs which are preferably used are those which allow the expression of the homogentisate phytyltransferase gene according to the invention in a host cell, also termed expression cassette hereinbelow.

The expression cassettes comprise regulatory nucleic acid sequences which govern the expression of the coding sequence in the host cell. In accordance with a preferred embodiment, an expression cassette encompasses, upstream, i.e. at the 5′ end of the coding sequencing, a promoter and downstream, i.e. at the 3′ end, a polyadenylation signal and, if appropriate, further regulatory elements linked functionally to the interposed coding sequence for the homogentisate phytyltransferase gene.

Functional linkage is to be understood as meaning the sequential arrangement of promoter, coding sequence, terminator and, if appropriate, further regulatory elements in such a manner that each of the regulatory elements can fulfill its intended function when the coding sequence is expressed. The sequences preferred for operative linkage, but not limited thereto, are targeting sequences for ensuring subcellular localization in the apoplast, in the vacuole, in plastids, in the mitochondrion, in the endoplasmatic reticulum (ER), in the nucleus, in elaioplasts or in other compartments, and translation enhancers such as the tobacco mosaic virus 5′ leader sequence (Gallie et al., Nucl. Acids Res. 15 (1987), 8693-8711).

Depending on the host organism or starting organism described in greater detail hereinbelow which is converted into a genetically modified or transgenic organism by introducing the expression cassette, different regulatory sequences are suitable.

Advantageous regulatory sequences for the nucleic acid constructs according to the invention, for the method described hereinbelow of producing vitamin E and for the genetically modified organisms described hereinbelow are, for example, present in promoters such as cos, tac, trp, tet, trp-tet, lpp, lac, lpp-lac, laciq, T7, T5, T3, gal, trc, ara, SP6, l-PR or in the l-PL promoter, all of which are advantageously used in Gram-negative bacteria.

Other advantageous regulatory sequences are present in, for example, the Gram-positive promoters amy and SPO2, in the yeast or fungal promoters ADC1, MFa, AC, P-60, CYC1, GAPDH, TEF, rp28, ADH or in the plant promoters CaMV/35S [Franck et al., Cell 21 (1980) 285-294], PRP1 [Ward et al., Plant. Mol. Biol. 22 (1993)], SSU, OCS, leb4, usp, STLS1, B33, nos or in the ubiquitin or phaseolin promoters.

As regards plants as genetically modified organisms, any promoter capable of governing the expression of foreign genes in plants is suitable in principle as promoter of the expression cassette.

A promoter which is preferably used is, in particular, a plant promoter or one which is derived from a plant virus. Especially preferred is the cauliflower mosaic virus CaMV 35S promoter (Franck et al., Cell 21 (1980), 285-294). As is known, this promoter comprises various recognition sequences for transcriptional effectors which, in their totality, lead to permanent and constitutive expression of the gene which has been inserted (Benfey et al., EMBO J. 8 (1989), 2195-2202).

The expression cassette can also comprise a pathogen-inducible or chemically inducible promoter by means of which expression of the exogenous homogentisate phytyltransferase gene in the plant can be governed at a particular point in time.

Examples of such promoters which can be used are, for example, the PRP1 promoter (Ward et al., Plant. Mol. Biol. 22 (1993), 361-366), a salicyclic-acid-inducible promoter (WO 95/19443), a benzenesulfonamide-inducible promoter (EP-A 388186), a tetracyclin-inducible promoter (Gatz et al., (1992) Plant J. 2, 397-404), an abscisic-acid-inducible promoter (EP-A 335528) or an ethanol- or cyclohexanone-inducible promoter (WO 93/21334).

Furthermore, preferred promoters are, in particular, those which ensure expression in tissues or plant organs in which, for example, the biosynthesis of tocopherol or its precursors takes place or in which the products are advantageously accumulated.

Promoters which must be mentioned in particular are those for the entire plant owing to constitutive expression, such as, for example, CaMV promoter, the Agrobacterium OCS promoter (octopine synthase), the Agrobacterium NOS promoter (nopaline synthase), the ubiquitin promoter, promoters of vacuolar ATPase subunits, or the promoter of a proline-rich protein from wheat (wheat WO 9113991)

Furthermore, promoters which must be mentioned in particular are those which ensure leaf-specific expression. Promoters which must be mentioned are the potato cytosolic FBPase promoter (WO 97/05900), the Rubisco (ribulose-1,5-bisphosphate carboylase) SSU (small subunit) promoter or the potato ST-LSI promoter (Stockhaus et al., EMBO J. 8 (1989), 2445-245).

Examples of further suitable promoters are:

specific promoters for tubers, storage roots or roots such as, for example the patatin promoter class I (B33), the potato cathepsin D inhibitor promoter, the starch synthase (GBSS1) promoter or the sporamin promoter,

fruit-specific promoters such as, for example, the tomato fruit-specific promoter (EP409625),

fruit-maturation-specific promoters such as, for example, the tomato fruit-maturation-specific promoter (WO 94/21794),

flower-specific promoters such as, for example, the phytoene synthase promoter (WO 92/16635) or the promoter of the P-rr gene (WO 98/22593) or

specific plastid or chromoplast promoters such as, for example, the RNA polymerase promoter (WO 97/06250).

Other promoters which can be used advantageously are the Glycine max phosphoribosyl pyrophosphate amidotransferase promoter (see also Genbank Accession Nummer U87999) or another nodia-specific promoter as described in EP 249676.

In principle, all natural promoters together with their regulatory sequences such as those mentioned above can be used for the process according to the invention. In addition, synthetic promoters can also be used advantageously.

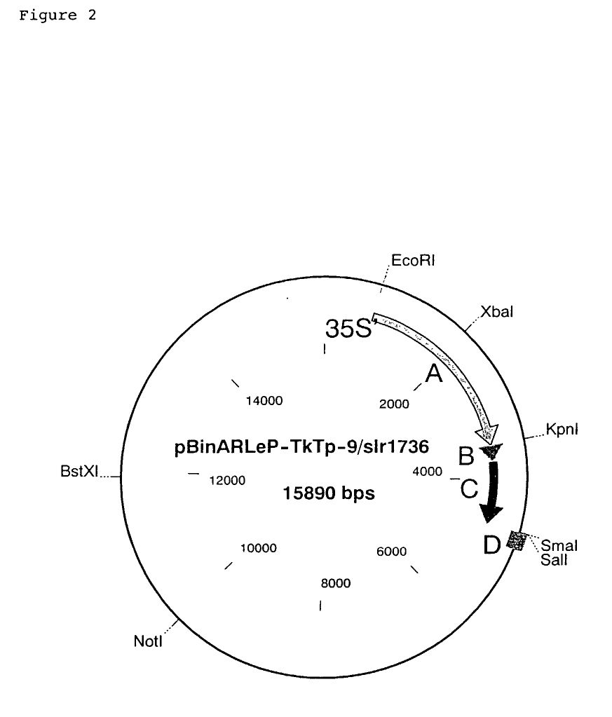

For example, the plant expression cassette can be incorporated into a derivative of the transformation vector pBin-19 with 35S promoter (Bevan, M., Nucleic Acids Research 12: 8711-8721 (1984)). FIG. 2 shows a derivative of the transformation vector pBin-19 with seed-specific legumin B4 promoter.

The expression cassette can comprise, for example, a seed-specific promoter (preferably the phaseolin promoter (U.S. Pat. No. 5,504,200), the USP promoter (Baumlein, H. et al., Mol. Gen. Genet. (1991) 225 (3), 459-467), the Brassica Bce4 gene promoter (WO 91/13980) or the LEB4 promoter (Fiedler and Conrad, 1995)), the LEB4 signal peptide, the gene to be expressed and an ER retention signal.

An expression cassette is generated for example by fusing a suitable promoter with a suitable HGPT DNA sequence and, preferably, a DNA which encodes a chloroplast-specific transit peptide and which is inserted between promoter and HGPT DNA sequence, and with a polyadenylation signal, using customary recombination and cloning techniques as they are described, for example in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley—Interscience (1987).

Especially preferred sequences are those which ensure targeting into the plastids.

It is also possible to use expression cassettes whose DNA sequence encodes, for example, an HGPT fusion protein, part of the fusion being a transit peptide which governs translocation of the polypeptide. Preferred are the chloroplast-specific transit peptides, which are cleaved enzymatically from the HGPT moiety after the HGPT gene has been translocated into the chloroplasts. Especially preferred is the transit peptide which is derived from the plastid Nicotiana tabacum transketolase or another transit peptide (for example the Rubisco small subunit transit peptide, or the ferredoxin NADP oxidoreductase transit peptide and also the isopentenyl pyrophosphate isomerase-2 transit peptide) or its functional equivalent.

Especially preferred are DNA sequences of three cassettes of the plastid transit peptide of the tobacco plastid transketolase in three reading frames as KpnI/BamHI fragments with an ATG codon in the NcoI cleavage site:

pTP09
Kpn I_GGTACCATGGCGTCTTCTTCTTCTCTCACTCTGTCTCAAGCTAT
CCTCTCTCGTTCTGTCCCTCGCCATGGCTCTGCCTCTTCTTCTCAACTTT
CCCCTTCTTCTCTCACTTTTTCCGGCCTTAAATCCAATCCCAATATCACC
ACCTCCCGCCGCCGTACTCCTTCCTCCGCCGCCGCCGCCGCCGTCGTAAG
GTCACCGGCGATTCGTGCCTCAGCTGCAACCGAAACCATAGAGAAAACTG
AGACTGCGGGATCC_BamHI
pTP10
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGCTGGATCC_BamHI
pTP11
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGGATCC_BamHI

The inserted nucleotide sequence encoding an HGPT can be generated synthetically or obtained naturally or comprise a mixture of synthetic and natural DNA components, or else be composed of various heterologous HGPT gene segments of various organisms. In general, synthetic nucleotide sequences are generated which have codons preferred by plants. These codons which are preferred by plants can be determined from codons with the highest protein frequency which are expressed in most of the plant species of interest. When preparing an expression cassette, various DNA fragments can be manipulated in order to obtain a nucleotide sequence which expediently reads in the correct direction and which is provided with a correct reading frame. To connect the DNA fragments to each other, adapters or linkers may be added to the fragments.

The promoter and terminator regions can expediently be provided, in the direction of transcription, with a linker or polylinker comprising one or more restriction sites for insertion of this sequence. As a rule, the linker has 1 to 10, in most cases 1 to 8, preferably 2 to 6, restriction sites. In general, the linker within the regulator regions has a size of less than 100 bp, frequently less than 60 bp, but at least 5 bp. The promoter can be native, or homologous, or else foreign, or heterologous, to the host plant. The expression cassette comprises, in the 5′-3′ direction of transcription, the promoter, a DNA sequence encoding an HGPT gene and a region for transcriptional termination. Various termination regions can be exchanged for one another as desired.

Manipulations which provide suitable restriction cleavage sites or which eliminate excess DNA or restriction cleavage sites may be employed. In-vitro mutagenesis, primer repair, restriction or ligation may be used in cases where insertions, deletions or substitutions such as, for example, transitions and transversions, are suitable. Complementary ends of the fragments may be provided for ligation in the case of suitable manipulations such as, for example, restriction, chewing-back or filling up overhangs for blunt ends.

Preferred polyadenylation signals are plant polyadenylation signals, preferably those which correspond essentially to Agrobacterium tumefaciens T-DNA polyadenylation signals, in particular those of gene 3 of the T-DNA (octopine synthase) of the Ti plasmid pTiACH5 (Gielen et al., EMBO J. 3 (1984), 835 et seq.) or functional equivalents.

Preferably, the fused expression cassette encoding an HGPT gene is cloned into a vector, for example pBin19, which is suitable for transforming Agrobacterium tumefaciens. Agrobacteria which are transformed with such a vector can then be used in the known manner for transforming plants, in particular crop plants, such as, for example, tobacco plants, for example by bathing scarified leaves or leaf sections in an agrobacterial solution and subsequently culturing them in suitable media. The transformation of plants by agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants can be regenerated in the known manner from the transformed cells of the scarified leaves or leaf sections, and these plants comprise a gene for expression of an HGPT gene which is integrated into the expression cassette.

The nucleic acid constructs according to the invention can be used for the generation of genetically modified organisms. The genetically modified organisms are generated by transforming the host organisms, hereinbelow also termed starting organisms, with a construct comprising the HGPT gene.

Starting or host organisms are to be understood as meaning prokaryotic or eukaryotic organisms such as, for example, microorganisms, mosses or plants. Preferred microorganisms are bacteria, yeasts, algae or fungi.

Preferred bacteria are bacteria of the genus Escherichia, Erwinia, Agrobacterium, Flavobacterium, Alcaligenes or Cyanobacteria of the genus Synechocystis.

Preferred yeasts are Candida, Saccharomyces, Hansenula or Pichia.

Preferred fungi are Aspergillus, Trichoderma, Ashbya, Neurospora, Fusarium or other fungi described in Indian Chem Engr. Section B. Vol 37, No 1,2 (1995) on page 15, Table 6.

Preferred algae are green algae such as, for example, algae of the genus Haematococcus, Phaedactylum tricornatum, Volvox or Dunaliella.

The invention relates to a genetically modified organism where the genetic modification of the gene expression, relative to a wild type, of a nucleic acid according to the invention

is increased in the event that the starting organism comprises a nucleic acid according to the invention or

is caused in the event that the starting organism does not contain a nucleic acid according to the invention.

The transgenic organisms comprising the HGPT gene according to the invention are capable of converting homogentisate derivatives and phytyl pyrophosphate derivatives into 2-methylphytylhydroquinone derivatives and/or homogentisate derivatives and geranylgeranyl pyrophosphate derivatives in 2-methylgeranylgeranylhydroquinone derivatives.

These organisms can be used for example for the above-described biotransformation.

Transgenic organisms comprising an exogenous HGPT gene according to the invention which already, in the form of the starting organisms, possess the biosynthesis genes for the production of vitamin E, such as, for example, plants or other photosynthetically active organisms such as, for example, cyanobacteria, mosses or algae, exhibit an increased tocopherol and/or tocotrienol content compared with the respective wild type.

The invention therefore relates to such a genetically modified organism according to the invention whose vitamin E content is increased over that of the wild type.

The present invention furthermore relates to the use of the HGPT according to the invention or of the HGPT genes according to the invention for the production of vitamin E in transgenic organisms.

Genetically modified organisms according to the invention, preferably plants whose vitamin E content is increased over that of the wild type, can be used for producing vitamin E.

The present invention therefore also relates to processes for the production of vitamin E by growing a genetically modified organism according to the invention, preferably a genetically modified plant according to the invention, whose vitamin E content is increased over that of the wild type, harvesting the organism and subsequently isolating the vitamin E compounds from the organism.

Genetically modified plants according to the invention with an increased vitamin E content and which can be consumed by humans and animals can also be used as foodstuffs or animal feeds, for example directly or after processing which is known per se.

Used in a preferred embodiment for the generation of organisms with an increased vitamin E content (tocopherols and/or tocotrienols) compared with that of the wild type are plants, not only as starting organisms but also, accordingly, as genetically modified organisms.

Examples of preferred plants are Tagetes, sunflowers, Arabidopsis, tobacco, red pepper, soybeans, tomatoes, aubergines, capsicums, carrots, potatoes, maize, saladings and cabbages, cereals, alfalfa, oats, barley, rye, wheat, Triticale, panic grasses, rice, lucerne, flax, cotton, hemp, Brassicaceae such as, for example, oilseed rape or canola, sugar beet, sugar cane, nut and grapevine species or wood species such as, for example, aspen or yew.

Especially preferred are Arabidopsis thaliana, Tagetes erecta, Brassica napus, Nicotiana tabacum, canola, potatoes and oil crops such as, for example, soybeans.

The invention furthermore relates to a method of generating genetically modified organisms by introducing a nucleic acid according to the invention or a nucleic acid construct according to the invention into the genome of the starting organism.

To transform a host organism such as, for example, a plant, with a DNA encoding an HGPT, an expression cassette is incorporated, as insertion, into a recombinant vector whose vector DNA can preferably comprise additional functional regulatory signals, for example sequences for replication or integration.

Suitable vectors for plants are described, inter alia, in “Methods in Plant Molecular Biology and Biotechnology” (CRC Press), chapter 6/7, pp. 71-119 (1993).

Using the above-cited recombination and cloning techniques, the expression cassettes can be cloned into suitable vectors which allow their replication, for example in E. coli. Examples of suitable cloning vectors are pBR332, pUC series, M13mp series and pACYC184. Binary vectors, which are capable of replicating both in E. coli and in agrobacteria, are especially suitable.

The invention furthermore relates to the use of an expression cassette comprising a DNA sequence SEQ ID No. 1 or a DNA sequence hybridizing therewith for the transformation of plants, plant cells, plant tissues or plant parts. The preferred purpose of the use is to increase the tocopherol and/or tocotrienol content of the plant.

Depending on the choice of the promoter, expression may take place specifically in the leaves, in the seeds, in the petals or in other parts of the plant. Such transgenic plants, their propagation material, their plant cells, plant tissue or plant parts are a further subject of the present invention.

In addition, the expression cassette may also be employed for transforming bacteria, cyanobacteria, yeasts, filamentous fungi, mosses and algae for the purpose of increasing the tocopherol and/or tocotrienol content.

The transfer of foreign genes into the genome of a plant is termed transformation. It exploits the above-described methods of transforming and regenerating plants from plant tissues or plant cells for transient or stable transformation. Suitable methods are protoplast transformation by polyethylene-glycol-induced DNA uptake, the biolistic method using the gene gun—the so-called particle bombardment method, electroporation, incubation of dry embryos in DNA-containing solution, microinjection and agrobacterium-mediated gene transfer. The abovementioned methods are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225). The construct to be expressed is preferably cloned into a vector which is suitable for the transformation of Agrobacterium tumefaciens, for example pBin19 (Bevan et al., Nucl. Acids Res. 12 (1984), 8711).

Agrobacteria transformed with an expression cassette can equally be used in a known manner for transforming plants, for example by bathing scarified leaves or leaf sections in an agrobacterial solution and subsequently growing them in suitable media.

An increased tocopherol or tocotrienol content means for the purposes of the present invention the artificially acquired ability of an increased biosynthesis performance of these compounds by functionally overexpressing an HGPT gene according to the invention in the plant over the nonrecombinant plant.

In this context, it is possible to increase both the tocopherol content and the tocotrienol content. It is preferred to increase the tocopherol content. However, under certain conditions, it is also possible that it is preferred to increase the tocotrienol content.

For example, the biosynthesis site of tocopherols is, inter alia, the leaf tissue, so that leaf-specific expression of the HGPT gene is meaningful. However, it is obvious that tocopherol biosynthesis need not be restricted to leaf tissue but can also take place in a tissue-specific manner in all other parts of the plant, in particular in fatty seeds.

In addition, constitutive expression of the exogenous HGPT gene is advantageous. On the other hand, inducible expression may also appear desirable.

Expression efficacy of the recombinantly expressed HGPT gene can be determined for example in vitro by shoot meristem propagation. Also, changes in the nature and level of the expression of the HGPT gene, and their effect on tocopherol biosynthesis performance, can be tested on test plants in greenhouse experiments.

The invention furthermore relates to transgenic plants, transformed with an expression cassette comprising an HGPT gene according to the invention, and to transgenic cells, tissue, parts and propagation material of such plants.

Preferred in this context are as mentioned above transgenic plants such as, for example, Tagetes, sunflowers, Arabidopsis, tobacco, red pepper, soybeans, tomatoes, aubergines, capsicums, carrots, potatoes, maize, saladings and cabbages, cereals, alfalfa, oats, barley, rye, wheat, Triticale, panic grasses, rice, lucerne, flax, cotton, hemp, Brassicaceae such as, for example, oilseed rape or canola, sugar beet, sugar cane, nut and grapevine species or wood species such as, for example, aspen or yew.

Plants for the purpose of the invention are monocotyledonous and dicotyledonous plants.

The invention furthermore relates to other photosynthetically active organisms transformed with an expression cassette comprising an HGPT gene according to the invention.

Overexpression, in a plant, of the gene sequence encoding an HGPT according to the invention also results in an increased resistance to HGPT inhibitors, in addition to the increased vitamin E content.

The invention therefore relates to a genetically modified organism according to the invention, preferably a genetically modified plant according to the invention, which exhibits resistance to homogentisate phytyltransferase inhibitors.

For example, the present invention successfully allows, in transgenic plants, the homogentisate phytyltransferase (HGPT) activity to be increased by overexpressing the HGPT gene according to the invention. This can be achieved, in principle, by expressing homologous or heterologous HGPT genes.

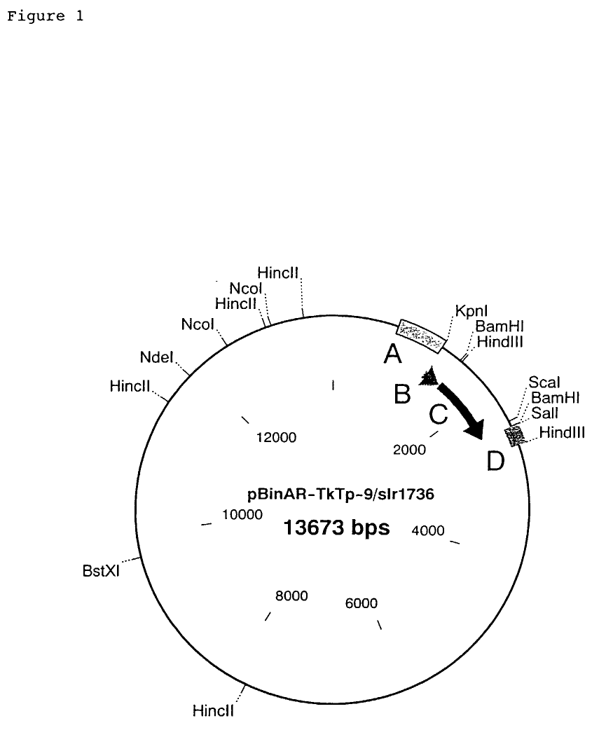

Example 1 describes for the first time cloning of an HGPT DNA sequence (SEQ ID No. 1) from Synechocystis spec. PCC 6803. To ensure localization in the plastids, a transit signal sequence (FIGS. 1-4) is placed upstream of the Synechocystis HGPT nucleotide sequence.

The 2-methylphytylhydroquinone and 2-methylgeranylgeranylhydroquinone, of which greater quantities are now available owing to the additional expression of the HGPT gene, are reacted further toward tocopherols and tocotrienol (FIG. 5).

Measurements on HGPT Synechocystis knock-out mutants reveal a drastic drop in the tocopherol content. This confirms the direct effect of the plastid plant HGPT on the synthesis of tocopherols and tocotrienols.

The invention furthermore relates to:

Methods of transforming a plant, wherein expression cassettes comprising an HGPT gene according to the invention are inserted into a plant cell or into plant protoplasts and are regenerated to give intact plants.

The use of the HGPT gene according to the invention for generating plants with an increased tocopherol and/or tocotrienol content by expressing, in plants, an HGPT DNA sequence.

The invention is now illustrated by the examples which follow, but is not limited thereto.

General Conditions:

Sequence Analysis of Recombinant DNA

Recombinant DNA molecules were sequenced using a Licor laser fluorescence DNA sequencer (available from MWG Biotech, Ebersbach) using the method of Sanger (Sanger et al., Proc. Natl. Acad. Sci. USA 74 (1977), 5463-5467).

### EXAMPLE 1

Cloning of the Synechocystis Spec. PCC 6803 Homogentisate Phytyltransferase.

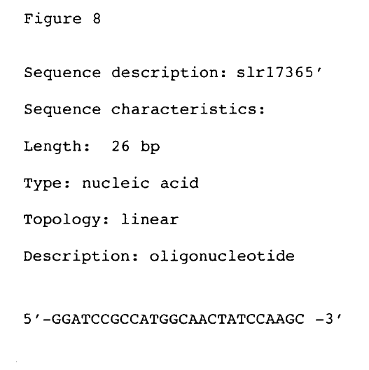

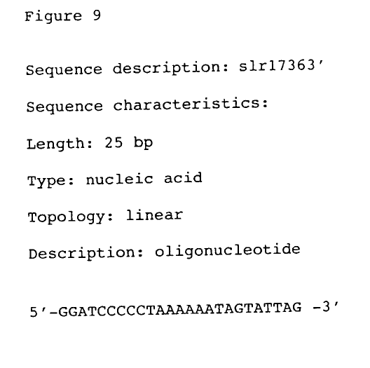

The DNA encoding the ORF slr1736 was amplified from Synechocystis spec. PCC 6803 by means of polymerase chain reaction (PCR) following the method of Crispin A. Howitt (BioTechniques 21:32-34, July 1996) using a sense-specific primer (slr17365′ FIG. 8, SEQ ID NO. 3) and an antisense-specific primer (slr17363′, FIG. 9, SEQ ID NO. 4).

The PCR conditions were as follows:

The PCR was carried out in a 50 μl reaction comprising:

5 μl of a Synechocystis spec. PCC 6803 cell suspension

0.2 mM dATP, dTTP, dGTP, dCTP

1.5 mM Mg (OAc)₂

5 μg bovine serum albumin

40 pmol slr17365′

40 pmol slr17363′

15 μl 3.3×rTth DNA polymerase XL buffer (PE Applied Biosystems)

5 U rTth DNA polymerase XL (PE Applied Biosystems)

The PCR was carried out under the following cycle conditions:

Step 1: 5 minutes at 94° C. (denaturation)

Step 2: 3 seconds at 94° C.

Step 3: 2 minutes at 48° C. (annealing)

Step 4: 2 minutes at 72° C. (elongation)

Steps 2-4 are repeated 35 times

Step 5: 10 minutes at 72° C. (post-elongation)

Step 6: 4° C. (waiting loop)

The amplicon was cloned into the PCR cloning vector pGEM-T (Promega) using standard methods. The identity of the amplicon generated was confirmed by sequencing using the M13F (−40) primer.

### EXAMPLE 2

Generation of an slr1736 knock-out Mutant.

A DNA construct for generating a deletion mutant of ORF slr1736 in Synechocystis spec. PCC 6803 was generated using standard cloning techniques.

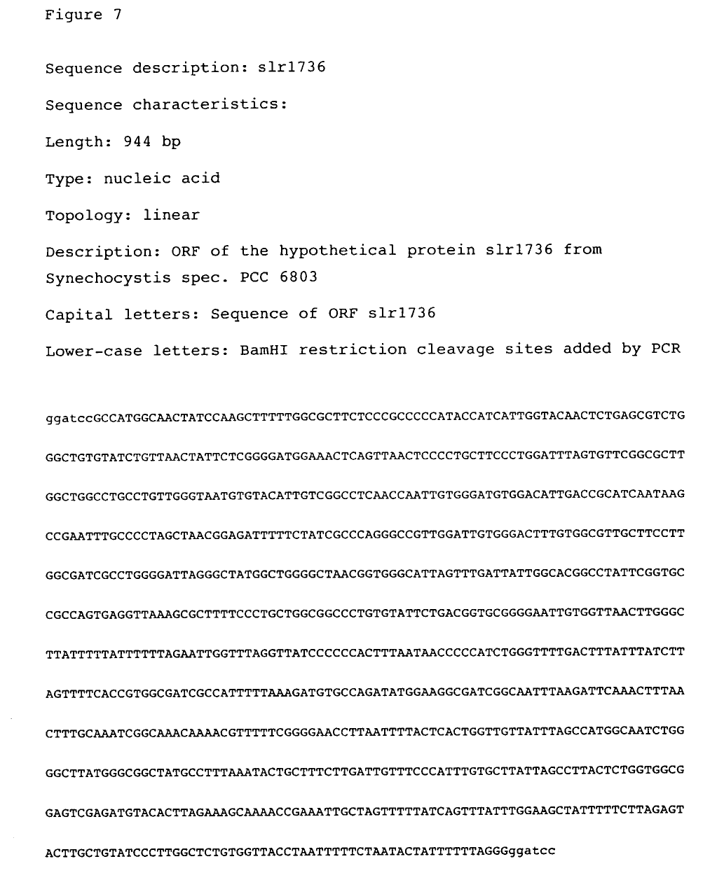

The vector pGEM-T/slr1736 was digested using the restriction enzyme HpaI. This digest deletes a 348 bp internal fragment of slr1736. The aminoglycoside 3′ phosphotransferase of the transposon Tn903 was subsequently cloned into the HpaI cleavage sites. To this end, Tn903 was isolated from the vector pUC4k (Vieira, J and Messing, J., Gene:19, 259-268, 1982) as EcoRI fragment, the overhangs of the restriction digest were made blunt-ended using standard methods and ligated into the HpaI cleaved vector pGEM-T/slr1736. The ligation was used for transforming E. coli ×11 blue cells. Transformants were selected by using kanamycin and ampicillin. A recombinant plasmid (pGEM-T/slr1736::tn903, see FIG. 6) was isolated and employed for the transformation of Synechocystis spec. PCC 6803 following the method of Williams (Methods Enzymol. 167:776-778, 1987).

FIG. 6 shows a construct for the knock-out mutagensis of ORF slr1736 in Synechocystis spec. PCC 6803.

Synechocystis spec. PCC 6803 transformants were selected on kanamycin-containing (km) solid BG-11 medium (Castenholz, Methods in Enzymology, 68-93, 1988) at 28° C. and 30 μmol photons ×(m²×s)⁻¹. After five selection cycles (passages of individual colonies to fresh BG-11 km medium), four independent knock-out mutants were generated.

The complete loss of the slr1736 endogene, or the exchange for the recombinant slr1736::tn903 DNA, was confirmed by PCR analyses.

### EXAMPLE 3

Comparison of the Tocopherol Production in Synechocystis Spec. PCC 6803 wild-type Cells and the Generated knock-out Mutants of ORF slr1736

The cells of the four independent Synechocystis spec. PCC 6803 knock-out mutants of ORF slr1736 which had been grown on the BG-11 km agar medium and untransformed wild-type cells were used to inoculate liquid cultures. These cultures were grown for approximately three days at 28° C. and 30 μmol photons×(m²×s)⁻¹ (30 μE). After determining the OD₇₃₀ of the individual cultures, the OD₇₃₀ of all cultures was synchronized by suitable dilutions with BG-11 (wild types) or BG-11 km (mutants). These cell-density-synchronized cultures were used to inoculate three cultures per mutant and the wild-type controls. Thus, the biochemical analyses were carried out using in each case three independently grown cultures of a mutant and the corresponding wild types. The cultures were grown to an optical density of OD₇₃₀=0.3. The cell culture medium was removed by two centrifugation steps at 14,000 rpm in an Eppendorf bench-top centrifuge. The cells were subsequently disrupted by four incubations for 15 minutes in an Eppendorf shaker at 30° C., 1000 rpm, in 100% methanol, and the supernatants obtained in each case were combined. Further incubation steps resulted in no further release of tocopherols or tocotrienols.

To avoid oxidation, the resulting extracts were analyzed directly after extraction with the aid of a Waters Alliance 2690 HPLC system. Tocopherols and tocotrienols were separated over a reversed-phase column (ProntoSil 200-3-C30, Bischoff) using a mobile phase of 100% methanol and identified with reference to standards (Merck). The detection system used was the fluorescence of the substances (excitation 295 nm, emission 320 nm), which was detected with the aid of a Jasco fluorescence detector FP 920.

No tocopherols were found in the Synechocystis spec. PCC 6803 knock-out mutants of the ORF slr1736. However, tocopherols were measured in the Synechocystis spec. PCC 6803 wild-type cells.

The loss of the ability of producing tocopherols within the knock-out mutants of ORF slr1736 compared with the Synechocystis spec. PCC 6803 wild-type cells shows that the gene slr1736 encodes a homogentisate phytyltransferase.

### EXAMPLE 4

Functional Characterization of the Synechocystis Spec. PCC6803 Homogentisate Phytyltransferase by Heterologous Expression in E. coli.

The hypothetical Synechocystis spec. PCC 6803 protein slr1736 was identified as homogentisate phytyltransferase by functional expression in E. coli.

The gene slr1736 which had been amplified from Synechocystis spec. PCC 6803 was subcloned into the expression vector pQE-30 (Qiagen) in the correct reading frame. The primers slr17365′ and slr17363′ (SEQ ID No. 2 and 3), which had been used for amplifying the ORF slr1736 from Synechocystis spec. PCC 6803 were constructed in such a way that BamHI restriction cleavage sites were added to the 5′-end and the 3′-end of the amplicon, see SEQ. ID No. 1. Using these flanking BamHI restriction cleavage sites, the slr1736 fragment was isolated from the recombinant plasmid pGEM-T/slr1736 and ligated into a BamHI-cut pQE-30 using standard methods. The ligation was used for the transformation of M15 E. coli cells, and kanamycin- and ampicillin-resistant transformants were analyzed. Kanamycin resistance is mediated by the plasmid pREP-4, which is present in the M15 cells. A recombinant plasmid (pQE-30/slr1736) which carried the slr1736 fragment in the correct orientation was isolated. Identity and orientation of the insert were confirmed by sequencing.

The recombinant plasmid pQE-30/slr1736 was used for transforming M15 E. coli cells in order to generate recombinant slr1736 protein. Using a colony which originated from the transformation, overnight culture in Luria broth medium supplemented with 200 μg/ml ampicillin (Amp) and 50 μg/ml kanamycin (Km) was inoculated. Starting from this culture, 100 ml Luria broth culture (Amp/Km) was inoculated the morning thereafter. This culture was incubated at 28° C. on a shaker-incubator until an OD₆₀₀ of 0.35-0.4 was reached. Then, production of the recombinant protein was induced by adding 0.4 mM isopropyl-β-D-thiogalactopyranoside (IPTG). The culture was shaken for a further 3 hours at 28° C., and the cells were subsequently pelleted by centrifugation at 8000 g.

The pellet was resuspended in 600 μl of lysis buffer (approx. 1-1.5 ml/g of pellet fresh weight, 10 mM HEPES KOH pH 7.8, 5 mM dithiothreinol (DTT), 0.24 M sorbitol). PMSF (phenyl methyl sulfonate) was subsequently added to a final concentration of 0.15 mM and the reaction was placed on ice for 10 minutes. The cells were disrupted by a 10-second ultrasound pulse using a sonifier. After addition of Triton×100 (final concentration 0.1%) the cell suspension was incubated on ice for 30 minutes. The mixture was subsequently spun down for 30 minutes at 25,000×g, and the supernatant employed in the assay.

The activity determination of the homogentisate phytyltransferase was carried out by detecting radiolabeled 2-methylphytylhydroquinone as reaction product.

To this end 235 μl of the enzyme (approx. 300-600 μg) together with 35 μl of phytyl pyrophosphate and 50 μl (1.2 nmol) of ³H homogentisic acid were incubated for 4 hours at 25° C. in the following reaction buffer: 100 μl (250 mM) Tricine-NaOH pH 7.6, 100 μl (1.25 mM) sorbitol, 10 μl (50 mM) MgCl₂ and 20 μl (250 mM) ascorbate. The tritium-labeled homogentisic acid was present in an ethanolic solution at 1 mg ascorbate/ml. Of this, 50 μl were concentrated, and the buffer and the enzyme and the phytyl pyrophosphate were added.

The reaction was quenched by extracting the batch twice using ethyl acetate. The ethyl acetate phases were concentrated and the residues taken up in methanol and applied to a thin-layer plate in order to separate the substances by chromatography (solid phase: HPTLC plates: silicagel 60 F₂₅₄ (Merck), liquid phase: toluene). The radiolabeled reaction product was detected by using a phosphoimager.

These experiments confirm that the protein encoded by the Synechocystis spec. PCC 6803 gene slr1736 (SEQ ID No.1) takes the form of a homogentisate phytyltransferase since it has the enzymatic activity for forming 2-methylphytylhydroquinone from homogentisate and phytyl pyrophosphate.

### EXAMPLE 5

Preparation of Expression Cassettes Comprising the HGPT Gene

Transgenic plants were generated which expressed the Synechocystis spec. PCC 6803 homogentisate phytyltransferase firstly under the control of the constitutive CaMV (cauliflower mosaic virus) 35S promoter (Franck et al., Cell 21: 285-294, 1980) and secondly under the control of the seed-specific promoter of the Vicia faba legumin gene (Kafatos et al., Nuc. Acid. Res., 14(6):2707-2720, 1986). The basis of the plasmid generated for the constitutive expression of the Synechocystis spec. PCC 6803 homogentisate phytyltransferase was pBinAR-TkTp-9 (Ralf Badur, PhD thesis, University of Göttingen, 1998). This vector is a derivative of pBinAR (Höfgen and Willmitzer, Plant Sci. 66: 221-230, 1990) and comprises the CaMV (cauliflower mosaic virus) 35S promoter (Franck et al., 1980), the termination signal of the octopine synthase gene (Gielen et al., EMBO J. 3: 835-846, 1984) and the DNA sequence encoding the transit peptide of the Nicotiana tabacum plastid transketolase. Cloning of the Synechocystis spec. PCC 6803 homogentisate phytyltransferase into this vector taking into consideration the correct reading frame generates a translational fusion of the homogentisate phytyltransferase to the plastid transit peptide. This causes the transgene to be transported into the plastids.

To construct this plasmid, gene slr1736 was isolated from plasmid pGEM-T/slr1736 using the flanking BamHI restriction cleavage sites. This fragment was ligated into a BamHI-cut pBinAR-TkTp-9 using standard methods (see FIG. 1). This plasmid (pBinAR-TkTp-9/slr1736) was used to generate transgenic Nicotiana tabacum plants.

Fragment A (529 bp) in FIG. 1 comprises the CaMV 35S promoter (nucleotides 6909 to 7437 of the cauliflower mosaic virus), fragment B (245 bp) encodes the transit peptide of the Nicotiana tabacum transketolase, fragment C (944 bp) encodes the

Synechocystis spec. PCC 6803 ORF slr1736, and fragment D (219 bp) encodes the termination signal of the octopine synthase gene.

The seed-specific promoter of the legumin B4 gene (Kafatos et al., Nuc. Acid. Res.,14(6):2707-2720, 1986) was used to generate a plasmid which allows the seed-specific expression of the Synechocystis spec. PCC 6803 homogentisate phytyltransferase in plants. The 2.7 kb fragment of the legumin B4 gene promoter was isolated from the plasmid pCR-Script/lePOCS, using the EcoRI cleavage site which flanks the promoter 5′ and the KpnI cleavage site which flanks the promoter 3′. The plasmid pBinAR-TkTp-9/slr1736 was also treated with the restriction enzymes EcoRI and KpnI. As a consequence, the CaMV 35S promoter was excised from this plasmid. The promoter of the legumin gene was subsequently cloned into this vector as EcoRI/KpnI fragment, thus generating a plasmid which placed the expression of the gene slr1736 under the control of this seed-specific promoter, see FIG. 2. This plasmid (pBinARleP-TkTp-9/slr1736) was used to generate transgenic Nicotiana tabacum plants.

Fragment A (2700 bp) in FIG. 2 contains the promoter of the Vicia faba legumin B4 gene, fragment B (245 bp) encodes the transit peptide of the Nicotiana tabacum transketolase, fragment C (944 bp) encodes the Synechocystis spec. PCC 6803 ORF slr1736, and fragment D (219 bp) the termination signal of the octopine synthase gene.

Generation of DNA constructs for expressing the Synechocystis spec. PCC 6803 homogentisate phytyltransferase in A. thaliana and B. napus.

To produce chimeric DNA constructs for the generation of transgenic A. thaliana or B. napus plants which express the Synechocystis spec. PCC 6803 homogentisate phytyltransferase, use was made of the vectors pPTVkan35S-IPP-Tp-9OCS and pPTVkanLeP-IPP-Tp-10NOS, respectively. These vectors are derivatives of pGPTVkan (D. Becker, E. Kemper, J. Schell, R. Masterson. Plant Molecular Biology 20: 1195-1197, 1992) whose uidA gene had been deleted. Instead, pPTVkan35S-IPP-Tp-9OCS contains the CaMV (cauliflower mosaic virus) 35S promoter (Franck et al., 1980), the sequence encoding the transit peptide of the A. thaliana plastid-specific isopentenyl pyrophosphate isomerase-2 (IPP-2) (Badur, unpublished) and the termination signal of the octopine synthase gene (Gielen et al., 1984)

The vector pPTVkanLeP-IPP-Tp-10nos contains the seed-specific promoter of the legumin B4 gene (Kafatos et al., Nuc. Acid.

Res.,14(6):2707-2720, 1986), also the sequence encoding the transit peptide of the A. thaliana plastid-specific isopentenyl pyrophosphate isomerase-2 (IPP-2) (Badur, unpublished) and the termination signal of the A. tumefaciens nopaline synthase (Depicker et al., J. Mol. Appl. Genet. 1, 561-73, 1982).

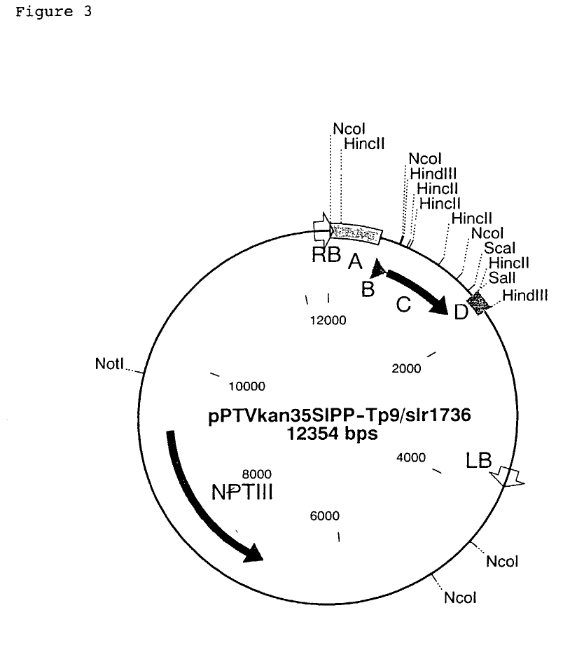

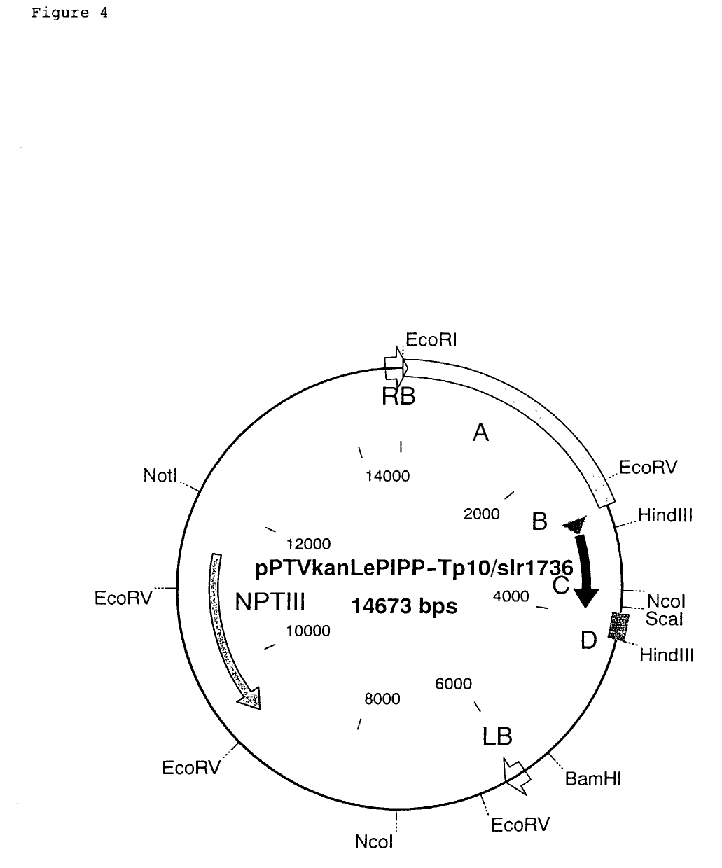

The DNA molecules encoding the Synechocystis spec. PCC 6803 ORF slr1736 were cloned into the vectors pPTVkan35S-IPP-Tp-9OCS (FIG. 3) and pPTVkanLeP-IPP-Tp-10nos (FIG. 4), respectively, in the form of BamHI fragments which had been made blunt-ended with T4 polymerase, thus generating a translational fusion with the IPP-2 transit peptide. Thus, the import of homogentisate phytyl-transferase into the plastids was ensured.

In FIG. 3, fragment A (529 bp) comprises the CaMV 35S promoter (nucleotides 6909 to 7437 of the cauliflower mosaic virus). Fragment B (205 bp) encodes the transit peptide of the A. thaliana isopentenyl pyrophosphate isomerase-2. Fragment C (944 bp) encodes the Synechocystis spec. PCC 6803 ORF slr1736. Fragment D (219 bp) encodes the termination signal of the octopine synthase gene.

In FIG. 4, fragment A (2700 bp) comprises the promoter of the Vicia faba legumin B4 gene, fragment B (206 bp) encoding the transit peptide of the A. thaliana isopentenyl pyrophosphate iso-merase-2. Fragment C (944 bp) encodes the Synechocystis spec. PCC 6803 ORF slr1736. Fragment D (272 bp) encodes the termination signal of the nopaline synthase gene.

### EXAMPLE 6

Generation of Transgenic Arabidopis thaliana Plants

Wild-type Arabidopsis thaliana plants (Columbia) were transformed with the Agrobacterium tumefaciens strain (GV3101 [pMP90]) on the basis of a modified vacuum infiltration method (Steve Clough and Andrew Bent. Floral dip: a simplified method for Agrobacterium mediated transformation of A. thaliana. Plant J 16(6):735-43, 1998; Bechtold, N. Ellis, J. and Pelltier, G., in: Planta Agrobacterium-mediated gene transfer by infiltration of adult Arabidopsis thaliana plants. CRAcad Sci Paris, 1993, 1144(2):204-212). The Agrobacterium tumefaciens cells used had previously been transformed with the plasmids pPTVkan35SIPP-Tp9/slr1736 and pPTVkanLePIPP-Tp9/slr1736 (FIGS. 3 and 4).

Seeds of the primary transformants were selected on the basis of their resistance to antibiotics. Seedlings which were resistant to antibiotics were planted into soil and the fully developed plants were used for biochemical analysis.

### EXAMPLE 7

Generation of Transgenic Brassica napus Plants

The generation of transgenic oilseed rape plants followed in principle a procedure of Bade, J. B. and Damm, B. (in Gene Transfer to Plants, Potrykus, I. and Spangenberg, G., eds, Springer Lab Manual, Springer Verlag, 1995, 30-38), which also indicates the composition of the media and buffers used.

The transformations were carried out with the Agrobacterium tumefaciens strain GV3101 [pMP90]. The plasmids pPTVkan35SIPP-Tp9/slr1736 and pPTVkanLePIPP-Tp10 /slr1736 (FIGS. 3 and 4) were used for the transformation. Seeds of Brassica napus var. Westar were surface-sterilized with 70% ethanol (v/v), washed for 10 minutes at 55° C. in water, incubated for 20 minutes in 1% strength hypochlorite solution (25% v/v Teepol, 0.1% v/v Tween 20) and washed six times with sterile water for in each case 20 minutes. The seeds were dried for three days on filter paper and 10-15 seeds were germinated in a glass flask containing 15 ml of germination medium. Roots and apices were removed from several seedlings (approx. size 10 cm), and the hypocotyls which remained were cut into sections of approx. length 6 mm. The approx. 600 explants thus obtained were washed for 30 minutes in 50 ml of basal medium and transferred into a 300 ml flask. After addition of 100 ml of callus induction medium, the cultures were incubated for 24 hours at 100 rpm.

An overnight culture of agrobacterial strain was set up in Luria broth medium supplemented with kanamycin (20 mg/l) at 29° C., and 2 ml of this were incubated in 50 ml of Luria broth medium without kanamycin for 4 hours at 29° C. until an OD₆₀₀ of 0.4-0.5 as reached. After the culture had been pelleted for 25 minutes at 2000 rpm, the cell pellet was resuspended in 25 ml of basal medium. The bacterial concentration of the solution was brought to an OD₆₀₀ of 0.3 by adding more basal medium.

The callus induction medium was removed from the oilseed rape explants using sterile pipettes, 50 ml of agrobacterial solution were added, and the reaction was mixed carefully and incubated for 20 minutes. The agrobacterial suspension was removed, the oilseed rape explants were washed for 1 minute with 50 ml of callus induction medium, and 100 ml of callus induction medium were subsequently added. Coculturing was carried out for 24 hours on an orbital shaker at 100 rpm. Coculturing was stopped by removing the callus induction medium and the explants were washed twice for in each case 1 minute with 25 ml and twice for 60 minutes with in each case 100 ml of wash medium at 100 rpm. The wash medium together with the explants was transferred into 15 cm Petri dishes, and the medium was removed using sterile pipettes.

For regeneration, in each case 20-30 explants were transferred into 90 mm Petri dishes containing 25 ml of shoot induction medium supplemented with kanamycin. The Petri dishes were sealed with 2 layers of Leukopor and incubated at 25° C. and 2000 lux at photoperiods of 16 hours light/8 hours darkness. Every 12 days, the calli which developed were transferred to fresh Petri dishes containing shoot induction medium. All further steps for the regeneration of intact plants were carried out as described by Bade, J. B and Damm, B. (in: Gene Transfer to Plants, Potrykus, I. and Spangenberg, G., eds, Springer Lab Manual, Springer Verlag, 1995, 30-38).

### EXAMPLE 8

Generation of Transgenic Nicotiana tabacum Plants

Ten ml of YEB medium supplemented with antibiotic (5 g/l beef extract, 1 g/l yeast extract, 5 g/l peptone, 5 g/l sucrose and 2 mM MgSO₄) were inoculated with a colony of Agrobacterium tumefaciens and the culture was grown overnight at 28° C. The cells were pelleted for 20 minutes at 4° C., 3500 rpm, using a bench-top centrifuge and then resuspended under sterile conditions in fresh YEB medium without antibiotics. The cell suspension was used for the transformation.

The sterile-grown wild-type plants were obtained by vegetative propagation. To this end, only the tip of the plant was cut off and transferred to fresh 2MS medium in a sterile preserving jar. As regards the rest of the plant, the hairs on the upper side of the leaves and the central veins of the leaves were removed. Using a razor blade, the leaves were cut into sections of approximate size 1 cm². The agrobacterial culture was transferred into a small Petri dish (diameter 2 cm). The leaf sections were briefly drawn through this solution and placed with the underside of the leaves on 2MS medium in Petri dishes (diameter 9 cm) in such a way that they touched the medium. After two days in the dark at 25° C., the explants were transferred to plates with callus induction medium and warmed at 28° C. in a controlled-environment cabinet. The medium had to be changed every 7-10 days. As soon as calli formed, the explants were transferred into sterile preserving jars onto shoot induction medium supplemented with claforan (0.6% BiTec-Agar (g/v), 2.0 mg/l zeatin ribose, 0.02 mg/l naphthylacetic acid, 0.02 mg/l of gibberellic acid, 0.25 g/ml claforan, 1.6% glucose (g/v) and 50 mg/l kanamycin). Organogenesis started after approximately one month and it was possible to cut off the shoots which had formed. The shoots were grown on 2MS medium supplemented with claforan and selection marker. As soon as substantial root ball had developed, it was possible to pot up the plants in seed compost.

### EXAMPLE 9

Characterization of Transgenic Plants

To confirm that expression of the Synechocystis spec. PCC 6803 homogentisate phytyl transferase affected vitamin E biosynthesis in the transgenic plants, the tocopherol and tocotrienol contents in leaves and seeds of the plants (Arabidopsis thaliana, Brassica napus and Nicotiana tabacum) which had been transformed with the above-described constructs were analyzed. To this end, the transgenic plants were grown in the greenhouse, and plants which express the gene encoding the Synechocystis spec. PCC 6803 homogentisate phytyltransferase were analyzed at Northern level. The tocopherol content and the tocotrienol content in leaves and seeds of these plants were determined. In all cases, the tocopherol or tocotrienol concentration in transgenic plants which additionally express a nucleic acid according to the invention is elevated in comparison with untransformed plants.

We claim:

1. A nucleic acid construct comprising a nucleic acid encoding a homogentisate phytyltransferase and a sequence encoding a plastid transit peptide, and functionally linked to one or more regulatory signals which ensure transcription and translation in prokaryotic or eukaryotic organisms.

2. A nucleic acid construct as claimed in claim 1, wherein the regulatory signals comprise one or more promoters which ensure transcription and translation in prokaryotic or eukaryotic organisms.

3. A genetically modified organism where the genetic modification of the gene expression, relative to a wild type, of a nucleic acid encoding a homogentisate phytyltransferase in plastids
is increased in the event that the starting organism comprises a nucleic acid encoding a homogentisate phytyltransferase or
is caused in the event that the starting organism does not contain a nucleic acid encoding a homogentisate phytyltransferase.

4. A genetically modified organism as claimed in claim 3, which comprises a nucleic acid construct as claimed in claim 1 or 2.

5. A genetically modified organism as claimed in claim 3 or 4, wherein the genetically modified organism exhibits an increased vitamin E content compared with the wild type.

6. A genetically modified organism as claimed in any of claims 3 to 5, wherein a eukaryotic organism is used as organism.

7. A genetically modified organism as claimed in claim 6, wherein a plant is used as eukaryotic organisms.

8. The use of a genetically modified organism as claimed in any of claims 3 to 7 for the production of vitamin E or for the biotransformation of homogentisate derivatives and phytyl pyrophosphate derivatives into 2-methylphytylhydroquinone derivatives or homogentisate derivatives and geranylgeranyl pyrophosphate derivatives into 2-methylgeranyl-geranylhydroquinone derivatives.

9. A method for the generation of genetically modified organisms as claimed in any of claims 3 to 7, wherein a nucleic acid construct as claimed in claim 1 or 2 is introduced into the genome of the starting organism.

10. The use of the nucleic acid construct as claimed in claim 1 or 2 for the generation of genetically modified organisms.

11. A process for the production of vitamin E, wherein an organism as claimed in any of claims 3 to 7 is grown, the organism is harvested and the vitamin E compounds are subsequently selected from the organism.

12. The use of a genetically modified organism as claimed in any of claims 3 to 7 as animal feeds and foodstuffs.
